(19) United States
(12) Patent Application Publication
(10) Pub. No.: US 2025/0174341 A1
(43) Pub. Date: May 29, 2025

(54) SYSTEMS AND METHODS FOR GENERATING CUSTOMIZED MAINTENANCE INSTRUCTIONS FOR BIOMEDICAL ENGINEERS

(71) Applicant: KONINKLIJKE PHILIPS N.V., EINDHOVEN (NL)

(72) Inventors: Lu WANG, EINDHOVEN (NL); Sauvik BHATTACHARYA, EINDHOVEN (NL); Milosh STOLIKJ, EINDHOVEN (NL); Qi GAO, EINDHOVEN (NL); Emanuele BASTIANELLI, EINDHOVEN (NL); Nicky PRONK, EINDHOVEN (NL)

(21) Appl. No.: 18/959,798

(22) Filed: Nov. 26, 2024

Related U.S. Application Data
(60) Provisional application No. 63/602,699, filed on Nov. 27, 2023.

(51) Int. Cl. G16H 40/40; G06Q 10/0631

(52) U.S. Cl. CPC G16H 40/40; G06Q 10/063112

(57) ABSTRACT

A non-transitory computer readable medium (26) stores a database (30) storing a plurality of resolved historical service cases and service manuals (32) for a plurality of medical devices (12). Instructions executable by at least one electronic processor (20) to perform a maintenance assistance method (100) include receiving information describing servicing to be performed on a medical device (12); determining one or more skills related to performing the servicing by comparing the received information with one or more of the historical service cases and/or service manuals; identifying a skill gap of a person who is to perform the servicing by comparing the determined one or more skills with a service record of the person; and outputting guidance (38) for performing the servicing wherein the guidance is based on the identified skill gap.

### CROSS REFERENCE TO RELATED APPLICATIONS

This application claims the benefit of U.S. Provisional Patent Application No. 63/602,699 filed Nov. 27, 2023. This application is hereby incorporated by reference herein.

### FIELD

The following relates generally to the medical device maintenance arts, medical imaging device maintenance arts, biomedical engineer skill determination arts, and related arts.

### BACKGROUND

Medical devices occasionally exhibit malfunction, which requires maintenance. Depending on the malfunction, the problem can sometimes be handled by a biomedical engineer who is already onsite. If the biomedical engineer has sufficient knowledge and skill, he or she can handle the case; however, if the biomedical engineer does not have the requisite skill or knowledge, the biomedical engineer will typically hand over the case to the vendor, which usually introduces additional waiting time (e.g., communication with vendors cost time).

In some medical device servicing arrangements, cases handled by biomedical engineers can be recorded in a Computerized Maintenance Management System (CMMS), which is typically separate from a Complaint Handling System of a vendor. This means that maintenance case records can be decentralized amongst various data sources, e.g., from vendors, engineers and/or hospitals.

Biomedical engineers often need to perform maintenance on new medical devices, new medical systems and new problems with demanding clinical use. They can struggle with developing and maintaining the right technical knowledge to resolve the issues. The technical level of biomedical engineers varies because everyone has different levels of experience in various domains (e.g., Operation Room, Intensive Care Unit room, etc.).

To control costs and maximize uptime, hospitals would like in-house biomedical engineers to perform maintenance on the medical devices at the hospital, rather than calling a vendor to perform the maintenance. In practice, however, many cases handled by a vendor could have been handled by in-house biomedical engineers.

There is a knowledge gap to be filled to efficiently utilize the skills of on-site biomedical engineers, thus reducing downtime for hospitals and reducing workload for vendors.

The following discloses certain improvements to overcome these problems and others.

### SUMMARY

In one aspect, a non-transitory computer readable medium stores a database storing a plurality of resolved historical service cases and service manuals for a plurality of medical devices. Instructions executable by at least one electronic processor to perform a maintenance assistance method include receiving information describing servicing to be performed on a medical device; determining one or more skills related to performing the servicing by comparing the received information with one or more of the historical service cases and/or service manuals; identifying a skill gap of a person who is to perform the servicing by comparing the determined one or more skills with a service record of the person; and outputting guidance for performing the servicing wherein the guidance is based on the identified skill gap.

In another aspect, a maintenance assistance system includes an electronic processor programmed to perform a maintenance assistance method including receiving information describing servicing to be performed on a medical device; determining one or more skills related to performing the servicing by comparing the received information with one or more historical service cases and/or service manuals; identifying a skill gap of a person who is to perform the servicing by comparing the determined one or more skills with a service record of the person; determining a likelihood that the person can perform the servicing based on the skill gap; and responsive to the likelihood being below a threshold, outputting guidance comprising a recommendation to initiate a service call for a third party to perform the servicing, or responsive to the likelihood being above the threshold, outputting the guidance comprising information compensating for the skill gap.

In another aspect, a maintenance assistance method includes a database storing a plurality of resolved historical service cases and service manual for a plurality of medical devices in a database; receiving information describing servicing to be performed on a medical device; determining one or more skills related to performing the servicing by comparing the received information with one or more historical service cases and/or service manuals; identifying a skill gap of a person who is to perform the servicing by comparing the determined one or more skills with a service record of the person, the skill gap comprising a difference between a known task that the service record of the person indicates the person has previously performed and a skill related to resolving the servicing comprising a new task that the service record of the person indicates the person has not previously performed; and outputting guidance for performing the servicing including an explanation of the difference between the known task and the new task.

One advantage resides in providing a system for more efficiently utilizing on-site biomedical engineers.

Another advantage resides in annotating maintenance instructions with explanations that are customized to the skills of a biomedical engineer, to assist the biomedical engineer to perform maintenance on a medical device.

Another advantage resides in selecting the most qualified biomedical engineers to perform maintenance on a medical device based on the determined skills of a plurality of Biomedical engineers.

Another advantage resides in educating biomedical engineers on servicing processes for medical devices.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a medical device maintenance assistance apparatus in accordance with the present disclosure.

FIG. 2 diagrammatically illustrates an embodiment of a medical device maintenance assistance method using the apparatus of FIG. 1.

FIG. 3 diagrammatically illustrates an embodiment of modules of the medical device assistance apparatus of FIG. 1.

FIG. 4 shows a representation of a database of the apparatus of FIG. 1.

### DETAILED DESCRIPTION

The following pertains to approaches for providing individualized assistance to hospital biomedical engineer (biomed) personnel in resolving imaging device problems. Vendor service engineers are sometimes called in to handle cases that could have been effectively and more economically handled in-house by a biomed. However, the biomed may lack confidence or sufficient assistance to resolve the problem.

In some disclosed approaches, when a customer contacts the vendor about a problem with an imaging device, a service call analyzer compares the problem description with a database of resolved historical service cases to determine the service skills needed to resolve the problem. Each service skill is translated to an instruction to perform a task. These instructions can be drawn, for example, from the historical service cases and/or service manuals.

Additionally, a biomed skills analyzer accesses information about the biomed's service record retrieved from a database. The skill services needed are compared with the biomed's skills to determine a likelihood that the biomed can complete the tasks required to resolve the service case. (If there are multiple biomeds available then the biomed skills analyzer can analyze each biomed's service record and select the most suitable biomed whose skills best align with the needed service skills). In another example, if a customer provides access to schedules of the biomeds, the selection of the biomed to resolve the problem can also be based on the biomeds that are currently available. In addition, the biomed skills analyzer can also forecast a best resolution for the problem by identifying a biomed that may become available in a time period that offsets the likelihood that a “less skilled” biomed could be able to fix the problem.

If there are skill gaps, such as where the biomed has an undesirably low likelihood of completing a task, then the instruction to perform that task is augmented by an explanation that is individualized to the skill gap of the biomed. For this purpose, a database of explanations is extracted from notes in the historical service case records or other sources. Various approaches such as textual entailment or other processes can be used to tailor the extracted content into readily understood explanations. There may be multiple explanations for a given instruction suitable for different skill levels. The explanations may be in the form of tips, admonishments (e.g., “Check the color of the cable before testing”), identification of distinctions from similar tasks with which the biomed record indicates the biomed is already familiar (e.g., “Warning: this calibration is different from the calibration you used in your previous service work on model <XXX>”), and so forth.

The resulting instructions with added explanations are pushed to the biomed via the biomed's cellphone, tablet computer, or other electronic device. The instructions and explanations may also be recorded at the vendor's database to maintain a record of the provided instructions. In some embodiments, the initially pushed instructions may be adaptively adjusted based on feedback from the biomed. For example, as the biomed is performing the maintenance in accord with the pushed instructions, the biomed may be uncertain about how to perform a certain instruction, and indicate this to the system by way of selecting a user input such as “Please provide more information on this step.” In response, the system adjusts the biomed's skills and the skill gap, and revises the augmented explanation based on the updated skill gap. Advantageously, this processing can employ the same processing as was used to generate the initially pushed instructions, with the biomed's skills now adjusted based on the feedback. Such adaptive adjustment may be useful, for example, to accommodate skill atrophy over time (e.g., the biomed's servicing record may indicate the biomed possesses the skill to perform a step but the biomed may have forgotten how to do the step), or because the biomed's record may fail to account for the biomed having received assistance from a more senior biomed in performing a step that formed the basis for the initial assessment of the biomed's skills.

In another example, the resulting instructions could alter the recommendations. For example, a less skilled biomed with some instructions may have a better chance of fixing the problem, which then is a better resource usage than waiting for the more skilled biomed to be on the schedule.

The following discusses the approach in terms of assisting a biomed, but the disclosed approach could also be applied to assist a third-party service provider (that is, a service engineer who is not a hospital in-house employee and also not a vendor service engineer). In addition, the disclosed system can also include cost modeling taking into account the skills, likelihood of success, trade-offs in service tasks across a fleet, availability, down-time, contract coverage, clinical workload, and so forth. The determination is then who should do the work given the cost analysis which includes the likelihood of success and any limitations, such as regulatory, safety, cybersecurity requirements. A cost model then can evaluate whether the additional learning materials will significantly impact the outcome prediction to see if there are opportunities to improve the cost model.

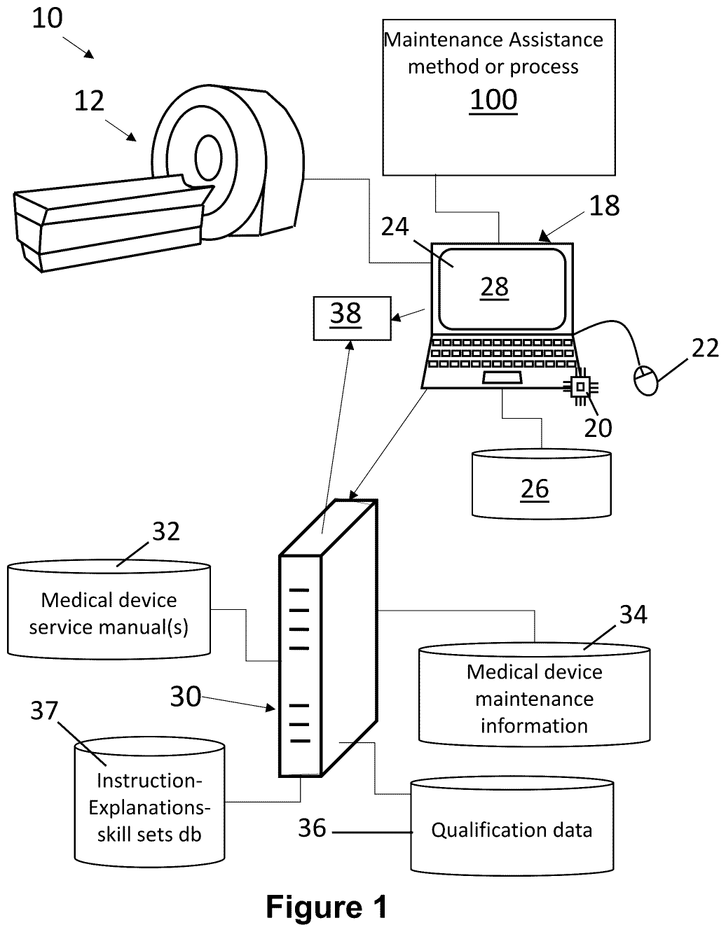

With reference to FIG. 1, an illustrative maintenance assistance system or apparatus 10 for guidance of maintenance of a medical device 12 is shown. The medical device 12, for example, can comprise an illustrative medical imaging device 12 (also referred to as a medical device, an imaging device, imaging scanner, and variants thereof) which can be a magnetic resonance imaging (MRI) scanner, a computed tomography (CT) scanner, a positron emission tomography (PET) scanner, a gamma camera for performing single-photon emission computed tomography (SPECT), an interventional radiology (IR) device, an X-ray device, an image-guided therapy (IGT) device, an ultrasound (US) device, or so forth. Although described herein as an imaging device, the medical device 12 can also be any other suitable medical device that is used with patients to perform medical functions such as diagnosis and/or treatment, such as a patient monitor, a radiation therapy device, a mechanical ventilator, a database, and so forth.

An electronic processing device 18, such as a workstation computer, or more generally a computer, a smart device (e.g., a cellular telephone (“cell phone”), a smart tablet, and so forth), is operable by a biomed. The electronic processing device 18 may also include a server computer or a plurality of server computers, e.g., interconnected to form a server cluster, cloud computing resource, or so forth, to perform more complex computational tasks. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18 or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The electronic processing device 18 is also in communication with a database 30 (shown in FIG. 1 as a server computer) that stores a plurality of service manuals 32 for a plurality of medical devices 12. For example, the service manuals 32 can include performance specifications (e.g., maximum slew rate of a gradient coil), physical dimensions, whether a component may contact a patient, and so forth. It will be appreciated that the number of service cases and service manuals 32 in the database 30 may be large, e.g., tens of thousands of cases, hundreds of thousands of cases, or more, and a search can in many cases return a few tens of thousands of specifications 32 (or more). In some embodiments, the database 30 further stores information on previously-performed resolved historical service cases. The database 30 also has includes or has access to medical device maintenance information 34 about the maintenance performed on the medical device 12 (and, typically, about maintenance performed on other medical devices in the hospital, radiology laboratory, or so forth). The medical device maintenance information 34 may, for example, include or be derived from a purchase record of components purchased for installation in the medical device 12 (for example, the purchase record may constitute or be extracted from a parts ordering system, parts inventory, or the like), a service record of the medical device 12, and/or the like. In the case of replacement of a component of the medical device 12 with a non-OEM component, the medical device maintenance information 34 may include a specification for the non-OEM replacement part obtained for example from the Internet (e.g., by accessing a manual for the non-OEM replacement part available at the supplier's website). In some cases, the non-OEM replacement part may include a QR code or bar code or the like which can be scanned using a cellphone or the like to automate retrieval of the specification information for the non-OEM part.

The apparatus 10 is configured as described above to perform a maintenance assistance method or process 100. The non-transitory storage medium 26 stores instructions which are readable and executable by the at least one electronic processor 20 to perform disclosed operations including performing the maintenance assistance method or process 100. In some examples, the method 100 may be performed at least in part by cloud processing.

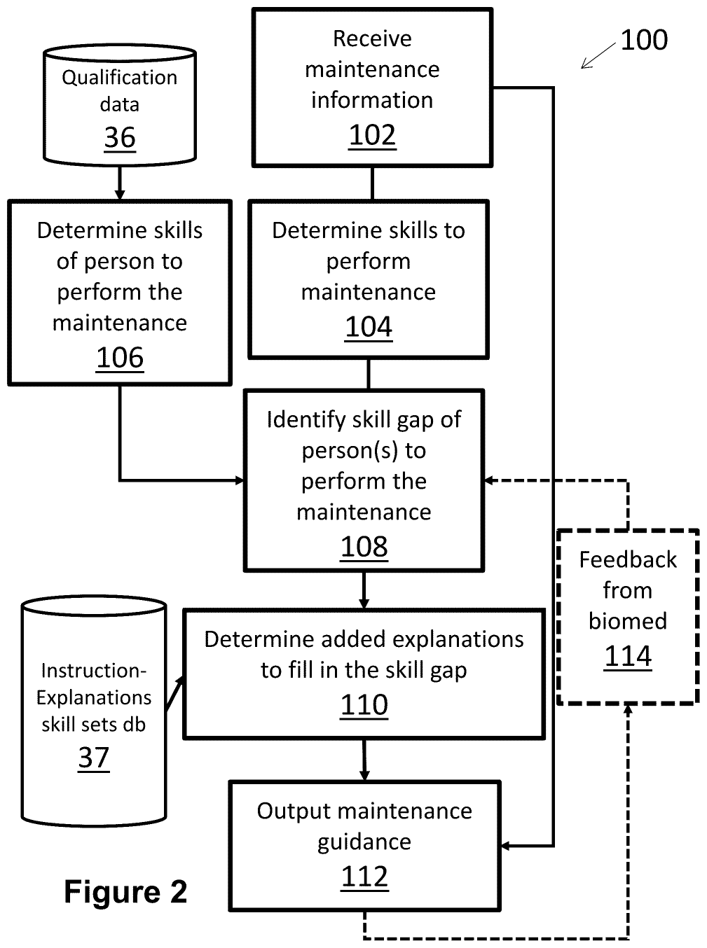

With reference to FIG. 2, and with continuing reference to FIG. 1, an illustrative embodiment of an instance of the method 100 is diagrammatically shown as a flowchart. At an operation 102, the information 34 describing servicing performed or planned to be performed on one or more of the medical devices 12 is retrieved from the database 30 and received at the electronic processing device 18. In some examples, the received information can include a replacement of an original equipment manufacturer (OEM) component of the medical device 12 with a non-OEM component or installation of a non-OEM software update or addition. In this example, the information received at the operation 102 may include specification information for the replacement non-OEM component. Such information may be retrieved from the Internet for example, e.g., using a search query automatically generated based on non-OEM component manufacturer and part number information, or obtained semi-automatically by scanning a QR code or bar code disposed on the non-OEM component. If the requisite information is not available in such an automated or semi-automated fashion, then the user may be asked to manually input relevant information on the non-OEM component to complete operation 102. The relevant information generally corresponds to analogous information for the medical device 12 set forth in the medical device service manuals 32, insofar as that information relates to the regulatory approval of the medical device. As one illustrative example, in the case of a medical imaging device the relevant information may include information such as attainable image resolution and other image quality characteristics, imaging field of view, maximum magnetic field applied to the patient (in the case of an MRI), whether a component may come into contact with the patient, and/or so forth.

At an operation 104, the electronic processing device 18 determines one or more skills related to performing the servicing by comparing the received servicing information 34 with one or more of the historical service cases and/or service manuals 32. Each skill corresponds to a task to be performed in the servicing. The skills are determined by a natural language processing operation (e.g., textual entailment) on the received information 34 to the historical service cases and/or service manuals 32.

At an operation 106, the skills of the person to perform the maintenance are determined. This may be done, for example, by referencing a database of qualification data 36 related to a plurality of bio-meds. Service records for a plurality of candidate persons available to perform the servicing are retrieved from the qualification data 36. For each candidate person, a skill gap of the candidate person is identified by comparing the determined one or more skills with the service record of the candidate person. The person who is to perform the servicing is selected as a candidate person having a smallest skill gap. The skills of the selected person are also determined from qualification data 36. For example, the qualification data 36 may include, for each candidate biomed, service records of previous servicing tasks performed by the biomed. From these service records, the steps that the biomed has previously performed are identified, and these suitably constitute the set of skills of the biomed.

Variant approaches can also be used, for example the biomed may be deemed to have a particular skill only if the service records for the biomed indicate the biomed has performed the task at least N times (where N could be an integer such as N=3, for example). In another variant, rather than the skills being binary values (i.e., the biomed has the skill or does not have the skill), the skills could be a percentage or scaled values. For example, if the biomed may be considered to have the skill at full level (e.g., 1.0) if the biomed's service records indicate the biomed has performed the task at least (for example) five times, and the skill level can be scaled downward for less experience. For example, if the biomed has performed the task only one, two, three, or four times then the skill level can be 0.2, 0.4, 0.6, 0.8, respectively. The skill level 0.0 is assigned if the biomed has never performed the task before according to the biomed's service records. These are merely non-limiting illustrative examples. Other sources of information can also optionally be utilized in the operation 106 to determine the skills of the biomed to perform the maintenance, such as educational or training records of the biomed, or a job title of the biomed (e.g., a senior biomed might be assumed to have certain skills, while a junior biomed might be assumed to lack certain skills a senior biomed would have).

In another example, if a customer provides access to schedules of the biomeds, the selection of the biomed to resolve the problem can also be based on the biomeds that are currently available. In addition, the biomed skills analyzer can also forecast a best resolution for the problem by identifying a biomed that may become available in a time period that offsets the likelihood that a “less skilled” biomed could be able to fix the problem.

In another example, the electronic processing device 18 can also perform cost modeling taking into account the skills of the biomeds, likelihood of success of the biomeds solving the problem, trade-offs in service tasks across a fleet, availability, down-time, contract coverage, clinical workload, and so forth. The determination is then which biomed should perform the servicing on the medical device 12 given the cost analysis which includes the likelihood of success and any limitations, such as regulatory, safety, cybersecurity requirements. A cost model then can evaluate whether the additional learning materials can significantly impact the outcome prediction to see if there are opportunities to improve the cost model.

At an operation 108, the electronic processing device 18 identifies a skill gap of the person (i.e., a bio-med or a third-party SE) who is to perform the servicing by comparing the determined one or more skills from the operation 104 with the skills of the person determined in the operation 106. The identifying of the skill gap includes identifying a difference between a known task that the service record of the person indicates the person has previously performed (determined in the operation 106) and a skill related to resolving the servicing (determined in the operation 104) comprising a new task that the service record of the person indicates the person has not satisfied a predetermined expertise threshold (i.e., the person has not previously performed the task, the person has not effectively performed the task before, the person has not performed the task often enough, and so forth).

In an operation 110, information compensating for the skill gap are determined and formulated as an explanation of the difference between the known task and the new task. For example, an instructions-explanations skill sets database 37 stores additional explanations for filling in skill gaps. The instruction explanations 37 may be generated by various approaches such as textual entailment or other NLP processing to tailor the gap information into readily understood explanations. There may be multiple explanations for a given instruction suitable for different skill levels or skill gaps. The explanations may be in the form of tips, admonishments, identification of distinctions from similar tasks with which the biomed record indicates the biomed is already familiar, and so forth.

At an operation 112, guidance 38 for performing the servicing wherein the guidance is based on the identified skill gap is output, for example on the display device 24 of the electronic processing device 18. The guidance comprises the maintenance information 102 supplemented by the explanations added by the operation 110.

In an optional feedback loop 114, the provided guidance may be interactive. The operation 106 determines the skills of the biomed. However, this is based on the service records of the biomed, and may not fully reflect the current skill set of the biomed. For example, if the service records show the biomed has performed a particular task in the past then the operation may assign that skill to the biomed. But if those previous performances of the task were a while ago then the biomed's skill in that task may have atrophied so that the biomed is no longer confident about performing the task. In another example, the service records may show the biomed has performed a particular task in the past, but in fact the task was performed by a superior rather than by the biomed, so that the biomed does not actually possess that skill. In such cases, the biomed can press a “Help” softkey on the display device 24 presenting the guidance 38, or operate some other feedback input, to indicate the biomed needs help with a task. As shown in FIG. 2, this feedback 114 is fed back to the operation 108 to update the skill gap to account for the fact that the biomed is not confident in performing the task. This then leads to the operation 110 providing further explanation from the database 37 to fill in this newly identified skill gap. In some cases, if based on the feedback a likelihood that the person can perform the servicing is determined to be below a threshold, then the guidance 38 is updated to present a recommendation to initiate a service call for a third party to perform the servicing.

More generally, although not shown in FIG. 2 in some cases if the operation 110 determines the biomed has too low likelihood of success, then the guidance 38 output in the operation 112 suitably comprises the recommendation to initiate a service call, and further comprises an explanation of the identified skill gap underlying this recommendation. The explanation is generated from the plurality of resolved historical service cases and service manuals 32. A textual entailment or a natural language processing operation on the received information 34 to generate the guidance 38. On the other hand, if the likelihood is above a threshold, the guidance 38 includes information compensating for the skill gap as previously described. In another example, the resulting guidance 38 could alter the recommendations. For example, a less skilled biomed with some instructions may have a better chance of fixing the problem, which then is a better resource usage than waiting for the more skilled biomed to be on the schedule.

### EXAMPLE

The following describes the apparatus 10 and the method 100 in more detail. The apparatus 10 is configured to automatically analyze required skills for a new service case, analyze the skills of the available biomedical engineers, analyze the skill gaps, and generate customized explanation text on top of the instructions describing the standard procedure. Before analyzing required skills, the method 100 also determines if the case can be handled by a biomedical engineer.

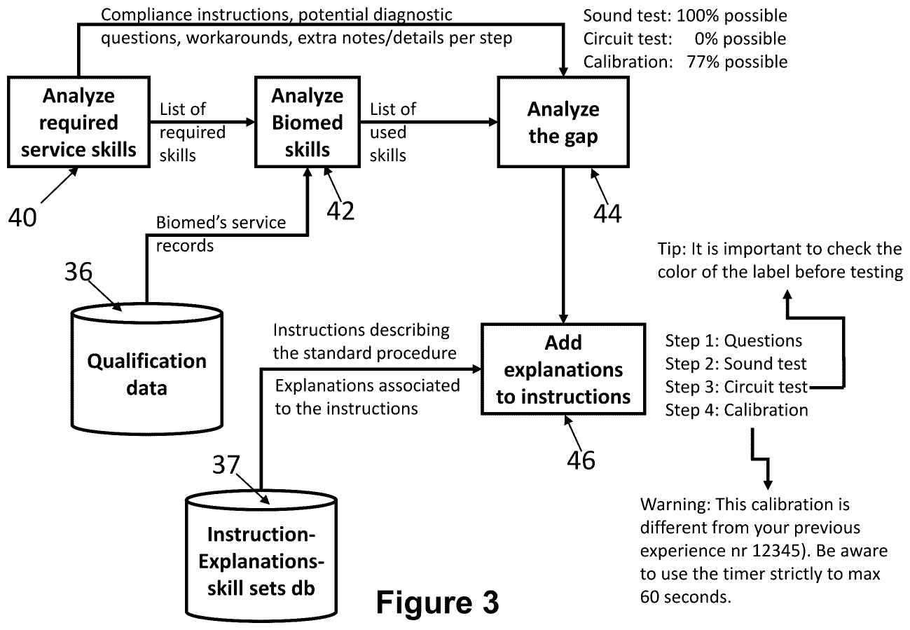

FIG. 3 shows another example of the apparatus 10. As shown in FIG. 3, the apparatus 10 includes a first module 40 implemented in the electronic processing device 18. The first module 40 is configured to analyze required service skills, for example, by analyzing the required skills based on the historic similar cases from service records that can likely be handled by a biomedical engineer or jointly with a remote SE from the vendor. To do so, the first module 40 is configured to retrospectively analyze similar cases solved by vendor's engineers and by Biomedical engineers. By looking into the service records, communications notes (which is part of the customer report), and other sources, it extracts the main skills and optionally level of experience needed to diagnose and repair it. For instance, for a case with error code ‘1243’ (no audio signal) on iXR model 1, the first module 40 retrieves about 100 historic similar cases that can be handled by a biomedical engineer. From all these 100 cases, the list of skills is aggregated. An example of output is listed in Table 1. The apparatus 10 can decide to include all the required skills retrieved or use statistical methods to select the only dominant skills.

TABLE 1
An example of the output of required skills of error ‘1243’ on IGT
model 1. The total number of retrieved similar historic cases is 100.

| Required | Required experience | Number of | Number of |
|---|---|---|---|
| skills | level (optional) | successful attempts | attempts |
| Sound test | Basic | 95 | 100 |
| Circuit test | Intermediate | 80 | 90 |
| Calibration | Basic | 50 | 77 |

In order to know what kind of cases can be handled by a biomedical engineer, a vendor or hospital creates a knowledgebase stating a list of diagnosis and parts replacement they would like the hospital Biomedical engineer to perform. If such a knowledgebase does not exist, it could be built by collecting engineers' input, (e.g., ‘Do you think this case could have been handled by the local biomedical engineer (yes/no?).’ In another example, similar cases from biomedical engineer service records are analyzed to determine what kind of cases can be handled by a biomedical engineer.

As shown in FIG. 3, the apparatus 10 includes a second module 42 implemented in the electronic processing device 18. The second module 42 is configured to analyze biomedical engineer skills, for example by analyzing the service experience of the biomedical engineer from service records 36 in detail. The output can be a list of devices, systems, tests and repairs the bio-med has worked with. To do so, the second module 42 is configured to analyze the service skill and optionally the experience of the particular biomedical engineer that is performing the maintenance activity. It retrieves all the cases handled by the biomedical engineer in the past and quantifies the skills and experience level of this biomedical engineer. The output can be a list of devices, systems, tests and repairs he has worked with. Table 2 is an example of the analysis of Biomedical engineer A, who has produced 200 case records.

TABLE 2
An example of the skills of biomedical engineer A
from the known service records.

| | | Number of | |
|---|---|---|---|
| | | successful | Number of |
| Devices/Systems | Skills | attempts | attempts |
| iXR model 1 | Sound test | 45 | 50 |
| iXR model 1 | Calibration | 2 | 10 |
| iXR model 2 | Sound test | 56 | 60 |

As shown in FIG. 3, the apparatus 10 includes a third module 44 implemented in the electronic processing device 18. The third module 44 is configured to analyze the skill gap, for example, by comparing the required skills of the current case with the skills of the biomedical engineers and computing the likelihood and the knowledge gap of the biomedical engineer. The output can be e.g., sound test 100% possible, circuit test 50% possible. The knowledge gap for Biomedical engineer A is the circuit test on XYZ steps.

From the output of the first and second modules 40, 42, and the given case (i.e., the given device/system type), the third module 44 compares the required skills with the existing skills of the Biomedical engineer. In a simple way, it sees each skill as a Boolean value (Yes/No). If a required sound test on iXR model1 is required, and there is any number of successfully resolved service records that the Biomedical engineer has done a sound test on iXR model 1, then this is a matching skill. In a more advanced way, it can compute the likelihood indicating how likely he matches with the required skill (output is Yes). This probability score can be used in the next unit to determine the level of detail on the extra explanations.

Here is one example of a more advanced way to analyze the gap. For a skill Y to match: Y=P (D, T, S, N1, N2) where P is the function to compute the likelihood indicating to what extent the Biomedical engineer skill matches with the required skill; D is the (compatible) device type or system type; T is the required skill for the device D; S is the existing skill or an adjacent skill; N1 is the number of successful attempts this skill S is used on the device; and N2 is the number of attempts this skill S is used on the device D.

Table 3 shows an example output of this unit. The likelihood of a 100% match can be in various ways. One way is to take the highest skilled engineer in the database as the 100% match. In case of a skill gap, a partner could be allocated.

TABLE 3
An example of the result of the gap analysis.

| Required Skills | Likelihood |
|---|---|
| Sound test | 100% |
| Calibration | 77% |
| Circuit test | 0% |

As shown in FIG. 3, the apparatus 10 includes a fourth module 46 implemented in the electronic processing device 18. The fourth module 46 is configured to add customized explanations to the guidance 38, for example, based on the procedure and analysis, add explanations, warnings and rationales to the steps in the procedure based on the previous analysis (e.g., if biomedical engineer A does not have experience with circuit tests, extra explanations and tips will be added on top of the instruction statement). The operation 46 suitably utilizes instruction explanation templates or information or the like contained in the database 37.

The fourth module 46 takes the output from the third module 44, depending on the gap, adds customized explanations to the guidance 38 describing the standard procedure. Depending on the level of matching, it can choose to add more (e.g., in case of low probability match) or less (e.g., in case of high probability match) details. Since this biomedical engineer is found to 100% match with the required skill ‘sound test as shown in Table 3’, no extra explanation will be added. Though the biomedical engineer has a good match with the skill ‘calibration,’ the method found out that the calibration test he performed before was on a different model, it points out the difference between performing calibration on the current model versus his previous experience. The explanation text could be ‘Warning, this calibration is different from your previous service (nr 12345). Be aware to use the timer strictly to max 60 s.’ For the skill ‘circuit test,’ it adds more explanations to the details because the Biomedical engineer had no experience before.

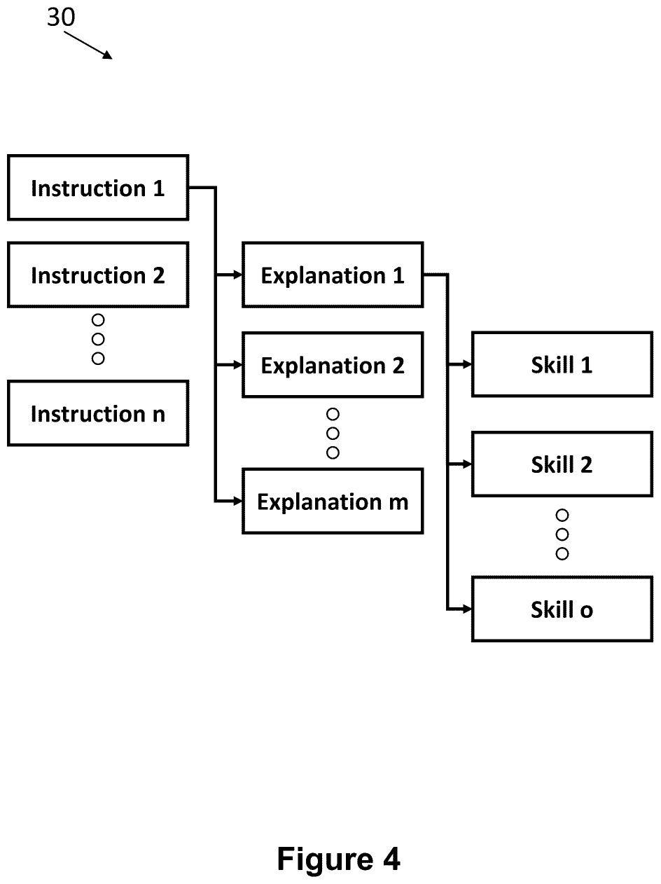

Referring now to FIG. 4, a schematic illustration of the generation of the guidance 38 is shown. The term “explanations” can include explanations, warnings, tips, practical notes etc. The explanations are retrieved from the database 30. For example, the database 30 can be organized as in FIG. 4. The database 30 comprises a set of instructions (I), a set of explanations (E), and a set of skills(S). Each instruction i is linked to a set of explanations E(i)={e}, and each explanation e is associated with a set of skills S(e)={s}. The association between the explanation and the skill set is interpreted as ‘the explanation is needed if the engineer does not have any one of the associated skills.

Then, for a given instruction i and engineer skill set S′, the system will retrieve all explanations eⱼ that are in E(i) and that the intersection between S (eⱼ) and Sⁱ is a non-empty set. This can be formulated as:

explanations(instruction i, engineer skills relevant for this case S_{c})={e|e∈E(i)∧(S_{c}∩S(e)≠Ø)}

where S_{c} indicates the set of matching skills from the third module 44.

In the example in FIG. 4, Instruction 1 can be ‘perform a circuit test’. The associated ‘Explanation l’ is ‘It's important to check the color of the cable before testing.’ The associated skill ‘skill 1’ to this explanation is ‘circuit test starter’. In the previous unit, this available Biomed has the circuit test skill on the starter level (see Table 3 circuit test matching 0%).

To build the instruction/explanation/skill database 30, first each instruction needs to be associated with related explanations. Such relationships can be built automatically in several ways. First of all, instruction and explanations may be present altogether in the notes of the same case. These can be extracted and related to each other according to the note structure. In absence of a detailed, parsable structure (e.g., technical questionnaires with answers), extracted instructions and explanations can be independently extracted from notes (or gathered through other sources, e.g., manually), and can be then associated through automatic methods. For example, textual similarity scores based on large language models, such as cosine similarity or BERTscore [see, e.g., Zhang, T., Kishore, V., Felix, W., Weinberger, K., & Artzi, Y. (2020). BERTScore: Evaluating Text Generation with {BERT}. 8th International Conference on Learning Representations (ICLR 2020). Addis Ababa, Ethiopia: OpenReview.net) can be used to get a value of the similarity between an instruction and an explanation. These can be used for one-to-one comparisons, together with some heuristics to decide which explanation goes with which instruction, or within more complex clustering algorithms.

A more advanced technique may rely on the so-called Textual Entailment task (see, e.g., Dagan, I., Roth, D., Sammons, M., & Zanzotto, F. (2013). Recognizing Textual Entailment: Models and Applications. Morgan & Claypool Publishers) (also referred to as Natural Language Inference or Sentence Entailment), where a model is trained on pair of sentences where one is supposed to entail the other. A similar type of relationship may hold between an instruction and an explanation. It is easy to build a model that performs this task, and existing trained models may be already used without the need of extra-training (see, e.g., Lan, Z., Chen, M., Goodman, S., Gimpel, K., Sharma, P., & Soricut, R. (2020). ALBERT: A Lite BERT for Self-supervised Learning of Language Representations. 8th International Conference on Learning Representations (ICLR 2020). Addis Ababa, Ethiopia: OpenReview.net). When fine-tuning or training is needed, a corpus of instructions paired with explanations must be collected. This can be done either by looking into the notes with the first two aforementioned approaches, with humans checking for the build pairs, or manually.

To associate explanation notes with the required skill level, the second module 42 extracts the case where the explanation comes from, together with other cases that have similar explanation notes, and computes the engineer's skill level as described in Table 2. For instance, the circuit test for iXR model 1, if the explanation note ‘check the color of the cable’ was found in 20 cases recorded by biomedical engineers, all corresponding skill levels of performing circuit test can be included with this explanation note, in this case, it is ‘circuit test starter’ and ‘circuit test beginner’. In a more advanced situation, when the explanation note is found in a service record from a vendor, the skill level of the vendor engineers can be computed the same way as in Table 2 and apply an extra factor to the skill level before it is associated with an explanation note because a vendor engineer may have higher level in general than a biomedical engineer. This factor could be pre-determined.

The disclosure has been described with reference to the preferred embodiments. Modifications and alterations may occur to others upon reading and understanding the preceding detailed description. It is intended that the exemplary embodiment be constructed as including all such modifications and alterations insofar as they come within the scope of the appended claims or the equivalents thereof.

1. A non-transitory computer readable medium storing:
a database storing a plurality of resolved historical service cases and service manuals for a plurality of medical devices; and
instructions executable by at least one electronic processor to perform a maintenance assistance method including:
receiving information describing servicing to be performed on a medical device;
determining one or more skills related to performing the servicing by comparing the received information with one or more of the historical service cases and/or service manuals;
identifying a skill gap of a person who is to perform the servicing by comparing the determined one or more skills with a service record of the person; and
outputting guidance for performing the servicing wherein the guidance is based on the identified skill gap.

2. The non-transitory computer readable medium of claim 1, wherein the database further stores qualification data related to a plurality of biomedical engineers, and the method further includes:
determining a likelihood that the person can perform the servicing based on the skill gap; and
if the likelihood is below a threshold, outputting the guidance comprising a recommendation to initiate a service call for a third party to perform the servicing.

3. The non-transitory computer readable medium of claim 2, wherein the guidance comprises the recommendation and further comprises an explanation of the skill gap.

4. The non-transitory computer readable medium of claim 3, wherein the explanation is generated from the plurality of resolved historical service cases and service manuals.

5. The non-transitory computer readable medium of claim 3, wherein the determining includes:
performing a natural language processing operation on the received information to generate the guidance.

6. The non-transitory computer readable medium of claim 1, wherein the database further stores qualification data related to a plurality of biomedical engineers, and the method further includes:
determining a likelihood that the person can perform the servicing based on the skill gap; and
if the likelihood is above a threshold, outputting the guidance comprising information compensating for the skill gap.

7. The non-transitory computer readable medium of claim 6, wherein:
the identifying of the skill gap includes identifying a difference between a known task that the service record of the person indicates the person has previously performed and a skill related to resolving the servicing comprising a new task that the service record of the person indicates the person has not satisfied a predetermined expertise threshold has not previously performed; and
the information compensating for the skill gap includes an explanation of the difference between the known task and the new task.

8. The non-transitory computer readable medium of claim 1, wherein the method further includes:
retrieving service records for a plurality of candidate persons available to perform the servicing;
for each candidate person, identifying a skill gap of the candidate person by comparing the determined one or more skills with the service record of the candidate person; and
selecting the person who is to perform the servicing as a candidate person having a smallest skill gap.

9. The non-transitory computer readable medium of claim 1, wherein each skill corresponds to a task to be performed in the servicing.

10. The non-transitory computer readable medium of claim 1, further comprising:
after outputting the guidance, receiving feedback from the person who is to perform the servicing;
adjusting the identified skill gap based on the feedback and outputting updated guidance for performing the servicing wherein the guidance is based on the adjusted identified skill gap.

11. A maintenance assistance system, comprising:
an electronic processor programmed to perform a maintenance assistance method including:
receiving information describing servicing to be performed on a medical device;
determining one or more skills related to performing the servicing by comparing the received information with one or more historical service cases and/or service manuals;
identifying a skill gap of a person who is to perform the servicing by comparing the determined one or more skills with a service record of the person;
determining a likelihood that the person can perform the servicing based on the skill gap; and
responsive to the likelihood being below a threshold, outputting guidance comprising a recommendation to initiate a service call for a third party to perform the servicing, or responsive to the likelihood being above the threshold, outputting the guidance comprising information compensating for the skill gap.

12. The system of claim 11, wherein the guidance comprises the recommendation and further comprises an explanation of the skill gap.

13. The system of claim 12, wherein the explanation is generated from the plurality of resolved historical service cases and service manuals.

14. The system of claim 12, wherein the determining includes:
performing a textual entailment or a natural language processing operation on the received information to generate the guidance.

15. The system of claim 11, wherein:
the identifying of the skill gap includes identifying a difference between a known task that the service record of the person indicates the person has previously performed and a skill related to resolving the servicing comprising a new task that the service record of the person indicates the person has not previously performed; and
the information compensating for the skill gap includes an explanation of the difference between the known task and the new task.

16. The system of claim 11, wherein the method further includes:
retrieving service records for a plurality of candidate persons available to perform the servicing;
for each candidate person, identifying a skill gap of the candidate person by comparing the determined one or more skills with the service record of the candidate person; and
selecting the person who is to perform the servicing as a candidate person having a smallest skill gap.

17. The system of claim 11, wherein each skill corresponds to a task to be performed in the servicing.

18. The system of claim 11, wherein the outputting includes:
outputting the guidance on an electronic processing device operable by a biomedical engineer.

19. A maintenance assistance method, comprising:
storing a plurality of resolved historical service cases and service manuals for a plurality of medical devices in a database;
receiving information describing servicing to be performed on a medical device;
determining one or more skills related to performing the servicing by comparing the received information with one or more historical service cases and/or service manuals;
identifying a skill gap of a person who is to perform the servicing by comparing the determined one or more skills with a service record of the person, the skill gap comprising a difference between a known task that the service record of the person indicates the person has previously performed and a skill related to resolving the servicing comprising a new task that the service record of the person indicates the person has not previously performed; and
outputting guidance for performing the servicing including an explanation of the difference between the known task and the new task.

20. The method of claim 19, further comprising:
responsive to the likelihood being below a threshold, outputting guidance comprising a recommendation to initiate a service call for a third party to perform the servicing, or responsive to the likelihood being above the threshold, outputting the guidance comprising information compensating for the skill gap.
